(19) United States
(12) Patent Application Publication
(10) Pub. No.: US 2006/0165749 A1
(43) Pub. Date: Jul. 27, 2006

(54) Moisture-activated release of fragrances from novel pourable lotion formulations

(76) Inventor: John J. Wille, Trenton, NJ (US)

Correspondence Address: LAW OFFICES OF NANCY A. BIRD, 231 WALTON AVENUE, SO. ORANGE, NJ, 07079, US

(21) Appl. No.: 11/339,419

(22) Filed: Jan. 25, 2006

Related U.S. Application Data
(60) Provisional application No. 60/646,896, filed on Jan. 25, 2005.

(51) Int. Cl. A01N 25/00; A61K 36/18; A61K 8/18; A61K 9/50; A61K 9/16

(52) U.S. Cl. 424/405; 424/725; 424/490; 424/65

(57) ABSTRACT

A method of sequestering volatile fragrances by encapsulating them in the oil phase of a thixotropic microemulsion, and drying to a thin film, and subsequently releasing the volatile oils upon rehydration of the dried film; compositions comprising the volatile fragrances encapsulated in the oil phase of a thixotropic microemulsion, which compositions may also contain temperature sensitive ingredients; and a wound dressing comprising a dried film formed from a thixotropic microemulsion.

### CROSS REFERENCE TO RELATED APPLICATIONS

This application claims priority from provisional Application 60/646,896 filed Jan. 25, 2005, entitled “Moisture-Activated Release of fragrances from a Novel Pourable Lotion Formulations”

### BACKGROUND OF THE INVENTION

The creation of cosmetic and personal care product formulations containing multiple ingredients presents many difficulties and challenges due to unanticipated behavior of any particular ingredient in the final formulation. One problem confronting formulators of fragrances is their instability upon prolonged product storage and or conversion to less stable forms during thermal processing of the final product formulation. Another concern is the interaction of stabilizing fixatives with other formulation ingredients. A still further concern is to provide for controlled and slow release of the desired fragrance after application to the desired body location.

The unique and unobvious formulations disclosed in this patent application deal with the above cited difficulties in formulating a stable and temporally-controlled release of fragrances through the application of a novel starch encapsulation technology. Encapsulation technology has received considerable attention for high volume applications such as household products, personal care, agriculture, packaging and coatings (Pothakamury U R and Barboa-Canovas G V. 1995. Trend in Food science & Technology 6: 397-406), including laundry products, cosmetics CR (controlled release) vitamins, CR probiotics, CR agrochemicals, CR plant hormones and antifungal compounds.

Environmental concerns over the large-scale use of solvents and cost associated with conventional matrix forming technologies such as solvent evaporation, emulsion encapsulants have prevented the use of these technologies for high volume applications. The objective of cost-effectiveness and efficiency can be accomplished by application of continuous processing techniques using an abundant matrix material to obtain encapsulation with tailored release properties. Starch is a widely employed matrix material. It possesses many favorable properties including abundance, low cost, processability, biodegradability and ease of chemical and enzymatic modification. It is capable of extrusion processing and encapsulation can be accomplished with heat sensitive compounds.

Starch-based delivery systems have been recently reviewed (Susan O. Freers, “Starch-based delivery systems, in Meyer R. Rosen (ed), Delivery System Handbook for Personal Care and Cosmetic Products, 741-760, 2005 William Andrew, Inc. In this review, the author discuss the various chemical and physical modification of starch that allow formulators to lessen retrograde gelation, attain controlled hydration, and means to lower the gelation temperature thus providing formulators ease and flexibility in processing conditions. Acid and enzymatic modifications of starch granules and chemical modification of starch granules have allowed cold processing, starch stabilization by radiation cross-linking, called “stabilized starches” have lowered gelation temperature and required less heat for hydration. Finally, starches can slightly oxidized by a variety of chemical oxidizing agents, including hydrogen peroxide, and sodium hypochlorite, which produce starches with lowered bioburden and improved adhesion at lower gelatinization temperatures.

Starch encapsulation of hydrophobic compounds was reported using a novel starch based matrix for encapsulation of heat sensitive compounds (Yilmaz G, ‘Novel starch based matrices for the encapsulation and controlled release of heat sensitive compounds prepared via melt extrusion technology,” J. Controlled Release Magazine, January, 2004). Other examples include the encapsulation of volatile compounds such as essential oils, flavors, perfumes, herbicides, pesticides, pheromones, vitamins, drugs, and bacterial cells (Yilmaz G, Jongboom R O J, Feil H, and Hennink W E, Carbohydrate Polmers, 45: 403410, 2001; Yilmaz G, Jongboom R O J, Feil H, and Hennik W E, Proc Intl Sympos Control Rel Bioact Mater., 2001; Yilmaz G, Jongboom R O J, Feil H, and Hennik, W E, “Encapsulation of living cells in starch using extrusion” Proc Intl Sym Control Rel Bioact Mater., 2002.

Earlier attempts to create an oil-in water emulsion include a jet cooking process for dispersing oil in starch gel was disclosed in Easkins and Fanta. U.S. Pat. No. 5,882,713 (1999), and Easkins and Fanta. U.S. Pat. No. 5,676,994 (1997). These patents describe methods to form a stable colloidal dispersion of oil in an aqueous starch matrix without the need for added emulsifiers. Stabilization of the starch-in-oil composite is due to a process in the steam jet cooking that deposits starch molecules at the oil-water interface around micron-sized oil droplets, encapsulating them (Eskins et al, 1996; Fanta and Eskins, 1998; Fanta et al, 1999; Fanta et al, 1999b).

The jet cooking process completely solubilizes the starch grains, which are then thoroughly mixed at elevated temperatures and steam pressures The turbulence so created disperses the oil droplets in the starch matrix. Further, the carbohydrate-coated oil droplets are unable to coalesce upon cooling, and the solubilized starch is largely prevented from undergoing retrograde gelation. The technology was trademarked as the Fantesk™ process. These inventors later reported that natural starches do contain small amounts of endogenous emulsifying agents including phospholipids and phosphoproteins, which may account for some of the emulsifying potential of high molecular weight natural starches. The technology has found applications in the food industry as thickeners and stabilizers, and in the water-proofing and sizing of paper.

The Fantesk™ technology has limited application in the personal care and cosmetic arena, due to its undesirable gel and film-forming properties. Indeed, natural starches have been passed over as cosmetic ingredients in favor of modified starches. As a consequence, attempts were made to develop a better technology to deliver cosmetically acceptable lotions and gels using natural starch and oils. This search was driven by the need to deliver natural products with hydrophobic properties and other hard to formulate plant-derived actives in a starch based formulation. It led to the Thixogel technology as disclosed by Wille, J J, U.S. patent application Ser. No. 10/873,590, 2005, entitled “Novel Topical Delivery System for Plant Derived Anti-Irritants.” The compositions and formulations revealed in that application based on oil-in-water emulsion system that does not depend on jet cooking, are simpler to process and results in a finer oil droplet size dispersion in a starch matrix.

The disclosed technology, described as Thixogel Technology, is defined as a thixotropic microemulsion. It was developed as a stable oil-in-water starch matrix. It has been differently_formulated to provide ease of formulating plant-derived hydrophobic anti-irritants. Most creams and lotions in the cosmetic market are emulsions. They may exist as microemulsions, multiple emulsions, fluorocarbon emulsions, and gel emulsions [Novel Cosmetic Delivery System, ed., Magdassi, S. Marcel Dekker, Inc., New York.

]. All Thixogel type formulations are O/W emulsions. More than a hundred Thixogel-based formulations have been made and tested to date (Wille, J J, “Thixogel™: Novel Topical Delivery System for Hydrophobic Plant Actives,” Meyer R. Rosen (ed). Delivery System Handbook for Personal Care and Cosmetic Products, 761-794, William Andrew, Inc, 2005). The basic ingredients of all Thixogel formulations are water, starch, oil, and an emulsifying agent. To this basic formula one may optionally include a humectant, one or more silicone type oils, an active agent such as an antimicrobial, hydrophobic plant actives, and natural preservatives. Among these four basic components many different natural and modified starches, many natural vegetable and synthetic oils, and anionic, cationic and non-ionic surfactants have been formulated. A common feature of all formulations is the stability of starch-oil dispersions formed by heating and mixing starch and oil under controlled temperature and mixing conditions. Thixogel formulations are so-called because they display thixotropic viscosity changes, i.e., they form semi-solids upon standing but with become pourable gels and lotions upon moderate mechanical agitation. Thixogel is a novel oil-in-water type hydrocolloid emulsion comprised of starch, water, oil, and an emulsifying surfactant. The starch may be a natural starch, or a modified starch. The oil may be paraffin oil, mineral oil, polydimethylsilicone oil, perflurofluorocarbon oil, vegetable oils, and various combinations of these oils. The emulsifying agent may be an anionic, a cationic or a non-ionic type of surfactant.

The present patent application discloses a novel and unexpected property of Thixogel starch-oil emulsions, which make them highly suitable for delivery of volatile ingredients such as essential oils, fragrances, flavors, perfumes, insectides, pheromones, and pesticides. The starch encapsulation of oil droplets containing volatile ingredients entraps the latter in a form that ensures their timed and controlled release. This occurs by moisture activation after application of the starch-oil emulsion to a substratum such as skin and the release of the entrapped volatile from the dried starch film upon exposure to conditions of low water moisture. This phenomenon is called “moisture-activation of fragrance release” which is triggered by hydration of dry starch films containing the oil-encapsulated volatiles.

### SUMMARY OF THE INVENTION

The compositions of the present invention may be used to formulate superior cosmetic and personal care products containing volatile components such as fragrances, essential oils, and aroma therapy oils. The present invention may also be employed for the controlled release of oil-soluble dermatological drugs, e.g. salicylic acid, hydrophobic vitamins, e.g., vitamin A and E, plant-derived botanical extracts and other hard to formulate hydrophobic cosmetic “actives.” In all such uses, upon application to skin, the drug-loaded compositions dehydrate to occlusive starch films containing active ingredients encapsulated in oil droplets oil, which active ingredients will be slowly released by water moisture trapped by the occlusive film, moisture subsequently applied to the dried film, and/or moisture due to transepidermal water loss. The compositions of the present invention may also be used to coat porous substrates, such as paper products, from which the volatile components may be released by the application of water. Compositions of the present invention can be stored in air sealed containers in the lotion state with a prolonged shelf life of many years.

The present application also includes methods of sequestering volatile oils in a thixotropic microemulsion, and in the dehydrated and/or dried films formed therefrom. Re-hydration of the dried films produces and instantaneous release of the volatile components. In addition, the methods of the present invention included subsequent release of fragrance by subsequent application of water; as well as redrying, followed by another release of fragrance, with water. The methods of the present application also include re-hydrating the dried film with water-based solution of temperature sensitive ingredients. This film may be dried to form a film with encapsulated volatile components, and temperature sensitive ingredients, which would not survive most encapsulation processes. The dried films may be milled to a fine powder, and later re-hydrated to form compositions, such as lotion compositions for topical application to the skin.

The compositions of the present invention also display thixotropic rheology changes, i.e., they form semi-solids upon standing but which become pourable gels and lotions upon moderate mechanical agitation, an important characteristic of a lotion or gel in that it minimizes dripping and provides easy application. Compositions of the present application also provide ease of spreading on skin and provide long-lasting protection against alcohol and water-based irritant chemicals.

Thixogel emulsions containing volatile fragrances are formed in a simple three-step process. Simply, a cornstarch slurry is prepared in cold water containing a specified low concentration of the surfactant and, if desired for moisturization, a specified amount of glycerol. The mixture is heated with continuous stirring until all of the starch is dissolved. The clarified starch is removed from heat, and allowed to cool to 65° C., when the oil phase ingredients are blended in the aqueous phase by low shear mechanical mixing. After a stable emulsion has formed oil-souble volatile components can be blended in when the melt has dropped below 55° C. Control of both temperature and mixing conditions are essential for reproducible and consistent formulation. Alternatively, the volatile components may be added after the oil phase ingredients have been mixed into the clarified starch.

Hydrocolloid gel emulsions containing volatile components are stable at least for three years at room temperature. The viscosity of such systems are a function of the starch to oil ratio. Low viscosity liquids are usually formed when the starch concentration is below 1% in such systems. Gels are seen at concentrations of starch in the range of 1% to 4%, and at oil concentrations below 10%. Starch-oil dispersions are achieved by processing at temperatures above 75° C. and are stabilized by low speed mechanical blending in the presence of low levels of a surfactant.

The stable oil-in-water thixotropic microemulsions of the present invention may be used to create a protective layer on the skin of the hands; a flexible, but invisible glove-type coating, or “glove-within-a glove.” The compositions of the present invention are alcohol resistant and moisturizing. A flexible yet, insensible film is formed by applying the moisture-activated fragrance release (MAFR) compositions of the present invention will tolerate multiple rinsing with 70% alcohol (ethanol), while maintaining the moisture of the underlying skin. Thus, the skin layer formed by the MAFR emulsion is a sanitary layer, which may be rinsed in alcohol. As may be easily understood, the MAFR-emulsions, when applied to the skin create a “wound dressing” skin layer, which may contain other added antimicrobial agents. In addition, a wound dressing may be formed from the dried starch film, with or without the volatile ingredients.

For a full understanding of the present invention, references should be made to the detailed description of the Thixogel Technology disclosed in the above cited U.S. Plant application Ser. No. 10/873,590 and PCTUS 05/21792, filed Jun. 22, 2005, and to the following detailed description of the invention in its preferred embodiments, and accompanying figures.

### BRIEF DESCRIPTION OF THE DRAWINGS

FIG. 1 is a photograph showing the effect of dehydrating and shrinkage of a starch-oil thixotropic microemulsion by exposure to 50% ethanol for 19 hours to form a dehydrated gel; and subsequently air drying of the dehydrated gel to a thin film.

FIG. 2 is a photograph showing the rapid restoration of the thin film of FIG. 1 to its original size before dehydration and air drying to a thin film, by addition of water to the dry film

FIG. 3 is a photograph of a wound dressing formed according to the present invention.

### DETAILED DESCRIPTION OF THE PREFERED EMBODIMENTS

### Example 1

### Moisture Activated Fragrance Release

It can be understood that there are many volatile fragrances available for moisture activated fragrance release from various Thixogel formulations. By way of example we have chosen two fragrances, Phenethyl alcohol (PEA) and Lavendar oil (LO). Typically, 0.5% PEA was incorporated into the oil phase ingredients of Formulations 1-5 (see EXAMPLE 5, List of Formulations). Alternatively, 0.5% PEA was introduced to the hot melt of the Thixogel starch gel microemulsions immediately after blending of the oil phase ingredients. Likewise, LO was either added directly into the oil phase ingredients prior to mixing with the aqueous phase ingredients or immediately after blending of the aqueous phase ingredient with the oil phase ingredients.

Fragrance Test:

Bibulous paper was cut into 1″ squares and impregnated with approximately 2 mg/ml of the fragrance-loaded Thixogel lotions. The impregnated papers were air-dried and stored in airtight sealed wrappers at room temperature under dehumidified conditions for varying lengths of time. To initiate fragrance release from the air-dried specimens, the impregnated papers were placed on dry paper toweling and small aliquots of water allowed to infiltrate the bibulous paper.

Results:

A panel of six blinded subjects were asked to smell a series of wetted bibulous papers 5 minutes after the papers were wetted. As a control, biblous papers were impregnated with vehicle lotions that did not have any added fragrances. Table 1 below present the results of this test panel.

TABLE 1
Sensory Assessment of PEA-Impregnated Fragrance Release
by Panel Subjects

| Subject No. | Control papers | PEA papers |
|---|---|---|
| 1 | No | Yes |
| 2 | No | Yes |
| 3 | No | Yes |
| 4 | No | Yes |
| 5 | No | Yes |
| Total 6 | All no | All yes |

Yes, detected the rose-like smell;
No-did not detect rose-like smell

In a second test, bibulous paper was impregnated with Lavendar Oil-containing Thixogel lotions and air-dried papers stored for 45 days. Again, a panel of six blinded subjects was asked to determine which of the wetted papers, control or PEA-imptegated samples gave off distinct rose-like fragrance. The results were identical to those disclosed in Table 1. All of the subjects correctly identified the rose-like fragrance only from the PEA-impregnated papers. Similar results were obtained from human panel studies using Lavendar oil containing Thixogel lotion impregnated papers versus un-impregnated controls. As a further control, bibulous papers were impregnated with 0.5% PEA or in oleophilic base containing Lavendar oil. When these papers were stored for 7 days or greater, no PEA-like or Lavendar oil-like scent could be detected by a panel of six blinded subjects.

Thus various the thixogel compositions with encapsulated volatile components may be impregnated into suitable porous substrates for the formation of various products. As an example, including inhalation release products may be formed with i.e. Camphor, or Menthol encapsulated into the thixogel compositions.

### Example 2

### Repeated Cycles of Fragrance Release from Once Impregnated Papers

In another study, bibulous papers were impregnated with PEA-containing Thixogel lotions allowed to air dry and stored under dehumidified conditions at room temperature for 7 days. On day 8, the papers were wetted with water and were found to release fragrance as predicted from the above results. The wetted papers were air dried again and stored for an additional 7 days. When rewetted with water these once-wetted papers again gave off a distinct rose-like fragrance indicative of moisture-activated fragrance release. This, too, was confirmed by a panel of six-blinded subjects.

In fact, fragrance release can be elicited repeatedly from the same piece of impregnated paper through several cycles of air-drying and moisture exposure.

Various other products requiring time release of fragrances, such as sachets, may be formed from the thixogel compositions with encapsulated volatile components impregnated into suitable porous substrates.

### Example 3

### Release of Fragrance from Skin

A Thixogel lotion formulation (Formulation 1) was loaded with 0.5% PEA an applied to the volar arm skin of several subjects. The lotion was allowed to dry on the skin for 30 minutes until no further scent could be detected. In all three subjects, scent could be restored by spraying a fine mist of water on the fragrance-treated skin areas.

### Example 4

### Effect of Other Releasing Agents Beside Water

Bibulous papers were impregnated with PEA-containing Thixogel lotion (Formulation 1) air dried and stored for 14 days. The fragrance test was conducted using different moisturizers as described in Table 2 below.

TABLE 2
Effect of Different Moisturizing Solvents on Fragrance Release
Solutions Prepared: 1)10% (v/v) of Propylene glycol(PG) in
water, 2) 14% (v/v) isopropyl alcohol (IP) in water, 3) 70% (v/v)
of ethanol in water (ETOH), and 4) pure deionized water.

| Solutions tested | Fragrance released^{a} |
|---|---|
| Water | Yes |
| 10% PG | Yes |
| 14% IP | Yes |
| 70% ETOH | No |

Fragrance was detected within 5 minutes after duplicate pieces of bibulous papers were wetted with the different solvents except IP which has its own scent. Once that had blown off the rose-like fragrance could be detected. Thus, typical water-based solvents may be used to accomplish the water release of the volatile components from the dried (or dehydrated) films.

### Example 5

### Fragrance Release from Dehydrated Gels and Powders

1. Preparation of Dehydrated and Rehydrated Gels

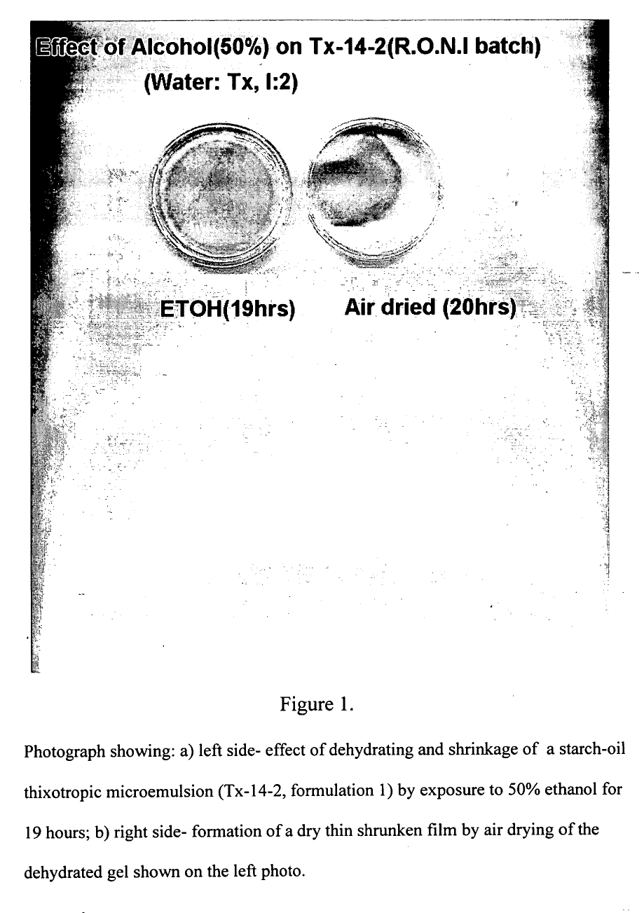

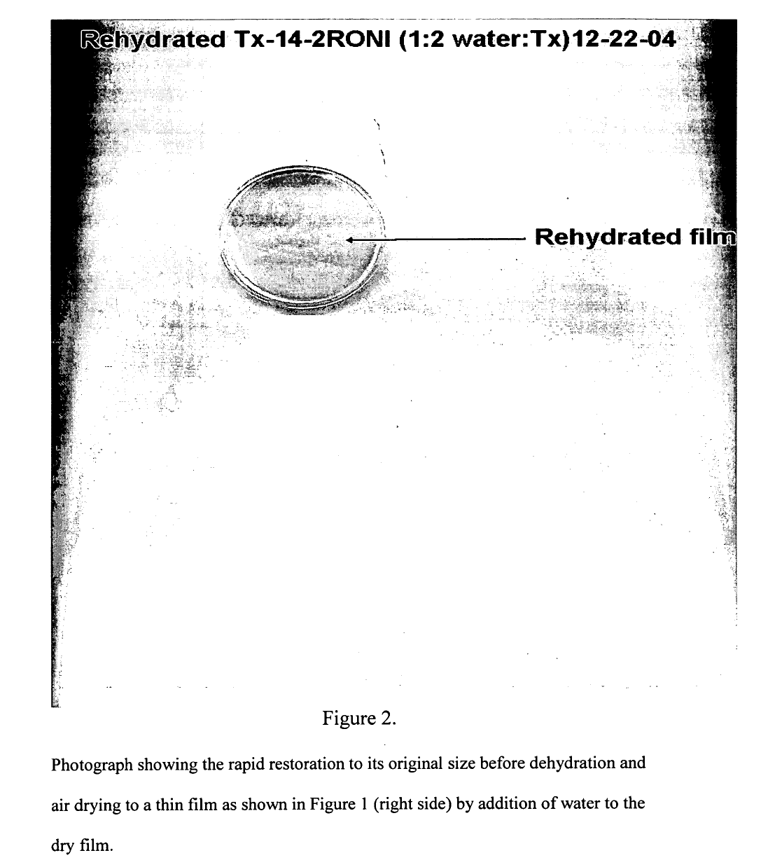

Formulation 1 was prepared and one volume of it was diluted with two volume of deinonized water. The diluted lotion/gel mixture was thoroughly mixed by stirring slowly under mild heating (60° C.) until a homogenously mixture was obtained. The diluted lotion containing about 1% starch and 3.3% oil; it was cast to a depth of 0.5 mm into a clean Petri dish and allowed to solidify into a solid gel overnight (about 20 hours) at room temperature. The solidified gels so formed are firm and non-pourable. In order to form a elastic gel the solidified cast gels were dehydrated by layering a sufficient amount of 50% Ethanol on top of the gelated surface for 19 hours. The gels undergo about 10% shrinkage in total surface area (see FIG. 1, left dish), and as a result of dehydration they are pliable and can be easily removed from the Petri dish with sterile forceps. When such dehydrated gels are removed to another glass Petri dish they can be further air-dried to a thin dry film that has less than 10% of the original weight of the original hydrated cast gel (see FIG. 1, right dish). Such dry and ethanol dehydrated gels will almost instantly rehydrate to their original weight when placed in a sufficient amount of distilled water (see FIG. 2).

This reversible hydration-dehydration process occurs without any appreciable loss of starch or oil. Further, it is possible to study fragrance release from dehydrated gels if one first loads the original ThixoDerm-F (Formulation 1) with a water-insoluble fragrance, such as PEA.

When, a dehydrated gel was prepared with ThixoDerm-F and assessed for its content of PEA, it was found that such dehydrated gels retain greater than 80% of the total concentration of PEA fragrance.

These results indicate that fragrance can be first encapsulated in the oil droplets in a starch matrix and that remains encapsulated even after alcohol dehydration and drying to a thin film. The technique of forming dry film with entrapped fragrances is a useful property for coating of artificial substrates, e.g., glossy paper for printing, from which a delayed release of fragrance is esteemed desirable. In addition, the dry films can be further processed to a powder by milling of the flaked films to a fine powder. Large scale processing of powders can be further accomplished by drum drying, flaking and milling with starch-oil composites that contain as much as 30% oils. “Instant” lotions may be formulated by rehydrating these powders to a lotion consistency.

2. Fragrance Release from Dehydrated Gels

Thin dry films of ThixoDerm-F were prepared as described above and were sprayed with a fine mist of water. A bloom of fragrance was readily detected within a few minutes of exposure to the water moisture.

3. Uptake of Water-Soluble Botanical Extracts at Ambient Temperatures.

Another application of the invention is the rehydration of dry films with aqueous solutions containing an active agent that is thermosensitive. The dry film will take up aqueous solutions of many water-soluble topical actives such as water-soluble botanical extracts that may lose much of their activity during heat processing steps required in the preparation of cosmetic emulsions. By contrast, dry Thixoderm The dried films can take up water-soluble antioxidants e.g., ascorbic acid (Vitamin C) without exposure to harsh conditions associated with heat processing, or emulsification with strong surfactants. The films may be subsequently dried, milled to a powder, and used to make “instant” lotions. Alternatively, the dried milled films described in 2 above may be rehydrated with a water-based solution containing the temperature sensitive ingredients, to form a composition, or an “instant” lotion, containing both encapsulated oil with volatile components and temperature sensitive ingredients which to not survive most encapsulation processes.

### Example 5A

### Wound Dressing

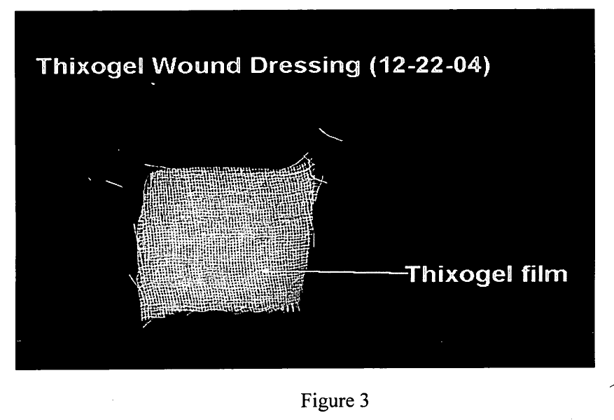

The dehydrated or dried films of the present invention, such as that shown in FIG. 1, right dish, may find use as wound dressings. The dried film can absorb up to 8× its weight in water, and can be used to absorb wound exudates. If desired the films may have typical wound healing ingredients incorporated therein, such as anti-microbials, or warming components such as camphor or for pain relief such as capsaicin. As the films are not skin adherent, it may be desirable to provide a backing, such as a nonwoven or woven fabric backing, which may be secured, as with an adhesive tape; or a film-type backing with an adhesive layer. Alternatively, the compositions of the present invention may be coated on the fabric backing before dehydrating or drying, to form a fibrous coating rather than a film. FIG. 3 illustrated wound dressing formed from a dried film made according to the present invention, and a gauze (fabric) backing.

### Example 6

### List of Formulations

Formulation 1.
THIXODERM-F: A Natural Emollient Fragrance Release Topical
Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid | | |
| | Mineral Oil | Oleophilic phase | 4.1 |
| | Berry Wax | Oleophilic phase | 9.3 |
| | EnviroPure310 ® | | |
| B | Deionized Water | Water phase (hydration) | 72.9 |
| | Corn Starch | Thickener (gel/ | 3.1 |
| | | film-forming agent) | |
| | Benzalkonium Chloride | Emulsifier | 0.5 |
| | Glycerol | Humectant | 5.0 |
| C. | CITRICIDAL | Natural preservative | 0.5 |
| D. | Phenethyl Alcohol | Fragrance | 0.5 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Olive Oil, EnviroPure310, React-NTI) and add directly to pre-heated Part B ingredients. Stir in Part C and D ingredient, and mix continuously until a homogeneous emulsion is formed. A variety of pure food grade comstarches cab be employed including Staley, National Starch, and Argo. This formulation is an example of a formulation with mixed oils. Formulations with mixed oils can accommodate a wider range of volatile components, and mixtures of volatile components, due to partitioning into the mixed oils.

Formulation 2
OLIVADERM: A Natural Emollient Topical Delivery System

| | Ingredient | Function(s) | Wt. % |
|---|---|---|---|
| A | Squalane | Oleophilic emollient | 7.5 |
| B | Deionized Water | Water phase (hydration) | 82.2 |
| | Corn Starch | Thickener (gel/ | 3.3 |
| | | film-forming agent) | |
| | Stepanquat NF 50 | Emulsifier | 1.0 |
| | (50% Benzalkonium Chloride) | | |
| | Glycerol | Humectant | 5.0 |
| C. | CITRICIDAL | Natural preservative | 0.5 |
| D. | Phenethyl Alcohol | Fragrance | 0.5 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat and cool to 65° C. Weight out Part A ingredient (Squalane, Vegetal) and add directly to pre-heated Part B ingredients. Stir in Part C and D ingredients, and mix continuously until a homogeneous emulsion is formed. A variety of pure food grade cornstarches can be employed including Staley, National Starch, and Argo. This formulation is an example of formulations utilizing oils, which are close to their melt point at room temperature, and thus produce better releases of fragrance from the oil phase.

Formulation 3
PolyDerm F - Fragrance Release Topical Gel Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 4.1 |
| | Petrolatum Jelly | Oleophilic phase | 7.5 |
| B | Deionized Water | Water phase (hydration) | 75.9 |
| | Guar Gum | Gel Thickener | 1.0 |
| | Benzalkonium Chloride | Emulsifier | 0.5 |
| | Glycerol | Humectant | 5.0 |
| C. | CITRICIDAL | Natural preservative | 0.5 |
| D. | Phenethyl Alcohol | Fragrance | 0.5 |

Weigh the Part B guar gum ingredient and place in suitable vessel equipped with mixer. Add a sufficient volume of deionized water, glycerol and benzalkonium chloride, mix thoroughly, and heat the Part B ingredients at 90° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

Formulation 4
SynDerm F - Fragrance Release Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 4.1 |
| | Petrolatum Jelly | Oleophilic phase | 9.3 |
| B | Deionized Water | Water phase (hydration) | 74.5 |
| | Carboxymethylcellulose | Thickener (gel forming agent) | 2.0 |
| | Benzalkonium Chloride | Emulsifier | 0.5 |
| | Glycerol | Humectant | 5.0 |
| C. | CITRICIDAL | Preservative | 0.5 |
| | Phenethyl Alcohol | Fragrance | 0.5 |

Weigh the Part B Carboxymethylcellulose, sodium salt (CMC) ingredient and place in suitable vessel equipped with mixer. Add a sufficient volume of deionized water, glycerol and benzalkonium chloride, mix thoroughly, and heat the Part B ingredients at 90° C. until the CMC is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

Formulation 5.
PolycelluDerm F-A Microcrystalline Cellulose Fragrance Releasing Gel
Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 4.1 |
| | Petrolatum Jelly | Oleophilic phase | 9.3 |
| B | Deionized Water | Water phase (hydration) | 72.2 |
| | Guar gum | Gel Thickener | 0.5 |
| | Cellulose/cellulose gum | Rheological modifier | 1.5 |
| | AVICEL 611(FMC) | | |
| | Benzalkonium Chloride | Emulsifier | 0.5 |
| | Glycerol | Humectant | 5.0 |
| C. | CITRICIDAL | Natural preservative | 0.5 |
| D. | Phenethyl Alcohol | Fragrance | 0.5 |

Weigh the Part B microcrystalline cellulose/cellulose gum (AVICEL 611, FMC) ingredient and place in suitable vessel equipped with mixer. Add a sufficient volume of deionized water, glycerol and benzalkonium chloride, mix thoroughly, and heat the Part B ingredients at 90° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

Formulation 6.
Berri-Seal F: A Natural Emollient Fragrance Releasing Topical Delivery
System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 4.1 |
| | Berry Wax | Oleophilic phase | 9.3 |
| | EnviroPure306 ® | | |
| B | Deionized Water | Water phase (hydration) | 73.5 |
| | Corn Starch | Thickener (gel/ | 3.1 |
| | | film-forming agent) | |
| | Glycerol | Humectant | 5.0 |
| C. | CITRICIDAL | Natural preservative | 0.5 |
| D. | Phenethyl Alcohol | Fragrance | 0.5 |

Weigh the Part B starch ingredient and place in suitable vessel equipped with mixer. Add a sufficient volume of deionized water, glycerol, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Soya and Canola oils, EnviroPure306, React-NTI) and add directly to pre-heated Part B ingredients. Stir in Part C and D ingredients, and mix continuously until a homogeneous emulsion is formed. A variety of pure food grade cornstarches can be employed including Staley, National Starch, and Argo. This formulation is an example of a formulation for ease of inclusion of higher molecular weight oils.

Formulation 7.
EVA/Oil-Seal F: A Natural Emollient Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 4.1 |
| | Ethylene Vinyl Acetate | Oleophilic phase | 9.3 |
| | EnviroPure301 ® | | |
| B | Deionized Water | Water phase (hydration) | 73.5 |
| | Corn Starch | Thickener (gel/ | 3.1 |
| | | film-forming agent) | |
| | Glycerol | Humectant | 5.0 |
| C. | CITRICIDAL | Natural preservative | 0.5 |
| D. | Phenethyl Alcohol | Fragrance | 0.5 |

Weigh the Part B starch ingredient and place in suitable vessel equipped with mixer. Add a sufficient volume of deionized water, glycerol, mix thoroughly, and heat the Part B ingredients at 90° C. until the starch is entirely dissolved. Remove from heat and cool to 65° C. Weight out Part A ingredient (mineral oil, DC-200, DC-245 and binder ethylene vinyl acetate/Soya and Canola oils, EnviroPure301, React-NTI) and add directly to pre-heated Part B ingredients. Stir in Part C and D ingredients, and mix continuously until a homogeneous emulsion is formed. A variety of pure food grade cornstarches can be employed including Staley, National Starch, and Argo. This formulation is an example of a formulation for ease of inclusion of lower melt point oils.

Formulation 8.
Berri/Olive Oil-Derm Seal: A Natural Emollient
Fragrance Releasing Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Olive Oil | Oleophilic phase | 3.9 |
| | Berry Wax | Oleophilic phase | 9.8 |
| | EnviroPure310 ® | | |
| B | Deionized Water | Water phase (hydration) | 72.5 |
| | Corn Starch | Thickener (gel/ | 3.3 |
| | | film-forming agent) | |
| | Benzalkonium Chloride | Surfactant | 0.5 |
| | Glycerol | Humectant | 5.0 |
| C. | CITRICIDAL | Natural preservative | 0.5 |
| D. | Phenethyl Alcohol | Fragrance | 0.5 |

Weigh the Part B starch ingredient and place in suitable vessel equipped with mixer. Add a sufficient volume of deionized water, glycerol, and benzalkonium chloride, mix thoroughly, and heat the Part B ingredients at 80° C. until the starch is entirely dissolved. Remove from heat and cool to 65° C. Weight out Part A ingredient (olive oil, DC-200, DC-245 and Berry Wax/Olive Oil, EnviroPure310, React-NTI) and add directly to pre-heated Part B ingredients. Stir in Part C and D ingredients, and mix continuously until a homogeneous emulsion is formed. A variety of pure food grade cornstarches cab be employed including Staley, National Starch, and Argo.

Formulation 9.
Modified Starch-Berri-Derm F: A Natural Emollient Fragrance
Releasing Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 3.2 |
| | DC-245 Fluid ® | | |
| | Mineral Oil | Oleophilic phase | 3.9 |
| | Berry Wax | Oleophilic phase | 9.8 |
| | EnviroPure306 ® | | |
| B | Deionized Water | Water phase (hydration) | 71.7 |
| | PureDent 836 Corn Starch | Thickener (gel/ | 3.3 |
| | | film-forming agent) | |
| | Benzalkonium Chloride | Surfactant | 0.4 |
| | Glycerol | Humectant | 5.0 |
| C. | CITRICIDAL | Natural preservative | 0.5 |
| D. | Phenethyl Alcohol | Fragrance | 0.5 |

Weigh the Part B PureDent 836 hydrophobically-modified corn starch (Grain Processing Corp., Muscatine, Iowa) ingredient and place in suitable vessel equipped with mixer. Add a sufficient volume of deionized water, glycerol, and benzalkonium chloride, mix thoroughly, and heat the Part B ingredients at 80° C. until the starch is entirely dissolved. Remove from heat and cool to 65° C. Weight out Part A ingredient (Mineral oil, DC-200, DC-245 and Berry Wax/Soya and Canola Oil, EnviroPure306, React-NTI) and add directly to pre-heated Part B ingredients. Stir in Part C and D ingredients, and mix continuously until a homogeneous emulsion is formed. This formulation is an example of a formulation using a starch, which can be cold mixed, and is desired for this property. However, these starches also partition the volatile components which are not encapsulated in the oil phase, yielding less control of the volatile components during processing, and less control of release of the volatile components on application of water (or water based solutions).

There has thus been shown and described a novel moisture-activated fragrance release property that resides in composition and formulations in the preferred embodiments of the present invention. It is to be understood, that the examples of fragrances employed in the above examples are not limited to those alone but can be any of the volatile components that that are generally useful as perfumes, scents and fragrances employed in cosmetic applications and familiar with the state of the art in cosmetic fragrance compositions. Many changes, modifications, variations and other uses and applications of the subject invention will, however, become apparent to those skilled in the art after considering the specification and the accompanying compositions and formulations which disclose the preferred embodiments thereof. All such changes, modifications, variations and other uses and applications which do not depart from the spirit and scope of the invention are deemed to be covered by the invention, which is not to be limited only by the claims which follow:

I claim:

1. A composition capable of moisture-activated fragrance release from oil-encapsulated starch films formed therefrom, said composition comprising a thixotropic microemulsion comprising:
i) about 1% to about 10% (w/v) starch,
ii) a cationic emulsifier,
iii) an oil from about 1% to about 10% (v/v), and
iv) a volatile fragrance,
said composition sequestering the volatile fragrance in the oil in the oil phase of the thixotropic microemulsion, and the starch films formed therefrom, and releasable from said starch films by exposure to water.

2. A composition as in claim 1, wherein the oil is an essential oil.

3. A composition as in claim 1, wherein the oil is an aroma therapy oil.

4. A topical formulation for application to the skin, comprising the composition of claim 1.

5. The topical formulation of claim 4 in an odor blocking product such as an anti-perspirant or deodorant.

6. The topical formulation of claim 4 in an insect repellant product.

7. A composition of claim 1, further comprising other aromatic oils, in the oil phase of the thixotropic microemulsion, which may also be encapsulated in the oil phase of the said oil-encapsulated starch films formed therefrom, and which are releasable from said starch films by exposure to water.

8. A method of sequestering a fragrance oil in a dried starch film and releasing the fragrance oil with water, said method comprising:
A) encapsulating a fragrance oil in the oil phase of a starch-oil dispersion system,
B) making a film of said system on a porous substrate,
C) drying the film to produce a fragrance indefinitely sequestered in the dried state as a oil-bearing starch film, and
D) hydrating the starch film with water to release the volatile fragrance, whereby the fragrance is emitted from the hydrated film almost instantaneously.

9. The method of claim 8, further comprising re-hydrating the starch film on a subsequent application of water, whereby a subsequent release of the volatile fragrance is emitted from the re-hydrated film almost instantaneously.

10. The method of claim 8, further comprising:
E) re-drying the starch film to sequester the volatile fragrance remaining therein, and
F) re-hydrating the starch film with water to release the volatile fragrance, whereby the fragrance is emitted from the re-hydrated film almost instantaneously

11. The method of claim 8, wherein the starch film is hydrated with a water-based solution of temperature sensitive ingredients.

12. The method of claim 11, further comprising
re-drying the starch film to sequester the volatile fragrance remaining therein, and
re-hydrating the starch film with water to release the volatile fragrance, whereby the fragrance is emitted from the re-hydrated film almost instantaneously

13. The method of claim 11, further comprising
re-drying the starch film to sequester the volatile fragrance remaining therein,
milling the dried film to a powder, and
re-hydrating the powder with water to form a lotion composition containing encapsulated volatile fragrance and temperature sensitive ingredients.

14. The method of claim 8, further comprising the step of dehydrating the film prior to drying the film to produce a fragrance indefinitely sequestered in the dried state as a oil-bearing starch film.

15. The method of claim 14, further comprising re-hydrating the starch film on a subsequent application of water, whereby a subsequent release of the volatile fragrance is emitted from the re-hydrated film almost instantaneously.

16. The method of claim 14, further comprising:
E) re-drying the starch film to sequester the volatile fragrance remaining therein, and
F) re-hydrating the starch film with water to release the volatile fragrance, whereby the fragrance is emitted from the re-hydrated film almost instantaneously

17. The method of claim 14, wherein the starch film is hydrated with a water-based solution of temperature sensitive ingredients.

18. The method of claim 17, further comprising
re-drying the starch film to sequester the volatile fragrance remaining therein, and
re-hydrating the starch film with water to release the volatile fragrance, whereby the fragrance is emitted from the re-hydrated film almost instantaneously

19. The method of claim 17, further comprising
re-drying the starch film to sequester the volatile fragrance remaining therein,
milling the dried film to a powder, and
re-hydrating the powder with water to form a lotion composition containing encapsulated volatile fragrance and temperature sensitive ingredients.

20. A wound dressing comprising a dried starch film formed by drying a 2:1 dilution of a thixotropic microemulsion comprising from about 1% to about 10% starch, a surfactant, and from about 1% to about 10% oil.
